(19) United States
(12) Patent Application Publication
(10) Pub. No.: US 2017/0304359 A1
(43) Pub. Date: Oct. 26, 2017

(54) ORAL IODINE DOSAGE FORM

(71) Applicant: BioPharmX, Inc., Menlo Park, CA (US)

(72) Inventors: Russell P. Elliott, Basking Ridge, NJ (US); Douglas W. Thomas, Palo Alto, CA (US); Akira Yamamoto, Cupertino, CA (US)

(21) Appl. No.: 15/494,405

(22) Filed: Apr. 21, 2017

Related U.S. Application Data
(60) Provisional application No. 62/325,982, filed on Apr. 21, 2016.

(51) Int. Cl. A61K 33/18; A61K 47/12; A61K 47/02; A61K 47/20; A61K 9/00

(52) U.S. Cl. CPC A61K 33/18; A61K 47/20; A61K 9/0053; A61K 47/02; A61K 47/12

(57) ABSTRACT

Provided is an oral dosage form comprising a deliverable form of iodine for treating symptoms related to fibrocystic breast condition, for prophylactically maintaining breast health, for treating fibrocystic breasts or breast cancer in pre-menopausal women, for prophylactically maintaining prostate health, and for treating benign prostate hyperplasia along with related methods for making and administering such dosage form. More particularly, this disclosure relates to an oral dosage form that is effective to deliver supraphysiologic levels of molecular iodine. The oral dosage form generally comprises a source of iodine and a reactive agent, wherein the source of iodine and/or the reactive agent are provided in excess.

### CROSS-REFERENCE TO RELATED APPLICATIONS

This application claims the benefit of priority to U.S. Provisional Application No. 62/325,982 filed Apr. 21, 2016, which is incorporated herein by reference in its entirety.

### FIELD

This disclosure relates generally to an oral dosage form comprising a deliverable form of iodine for treating symptoms related to fibrocystic breast condition and for prophylactically maintaining breast health, for treating fibrocystic breasts in pre-menopausal women, for treating breast cancer, for prophylactically maintaining prostate health, and for treating benign prostate hyperplasia along with related methods for making and administering such a dosage form. More particularly, this disclosure relates to an oral dosage form that is effective to deliver supraphysiologic levels of molecular iodine.

### BACKGROUND

Fibrocystic breast condition, also referred to as mammary dysplasia, benign breast condition, fibrocystic breast disease, and diffuse cystic mastopathy, is a benign condition, generally in pre-menopausal women, characterized by the presence of lumps or cysts in the breasts. The condition is fairly common; approximately 60% of women are estimated to have experienced fibrocystic breast condition at some point in their lives. The condition may, for example, be accompanied by pain or discomfort in one or both breasts, tenderness, and swelling. The condition can cause extreme discomfort, and can also make the detection of breast cancer more difficult due to changes in breast density.

The specific cause of fibrocystic breast condition and accompanying changes in the breasts isn't fully understood, although it is believed that estrogen and other reproductive hormones play a role. The occurrence of symptoms may be cyclical or non-cyclical (many women experience symptoms just prior to menstruation). Breast changes may be as a result of the change of diet over time. This link to dietary change has been reinforced by evidence that in Japan, where there is 25 times the consumption of iodine, there is a very low level of benign breast disease (Aceves C, Anguiano B, and Del G. “The Extrathyronine Actions of Iodine as Antioxidant, Apoptotic, and Differentiation Factor in Various Tissues” Thyroid (2013) 23(8): 938-946).

At present, there does not appear to be consensus amongst the members of the medical community regarding effective therapies for treating fibrocystic breast condition. Commonly recommended treatments include prophylactic administration of over-the-counter pain relievers such as ibuprofen or acetaminophen, application of warm or cold compresses, administration of estrogen or other hormones, administration of danazol, fine-needle aspiration for removing fluid from fluid-filled cysts or surgical removal if warranted, and modification of diet. Additional alternative approaches include the implementing a vegetarian diet, limiting the intake of caffeine, implementing a low-fat diet, and taking herbal remedies such as dandelion leaf, cleavers, and yarrow. Additional alternative approaches include the application of poke root oil or a gel or cream of natural progesterone to the breasts.

Another condition for which iodine is useful is treatment of prostate conditions and diseases, such as benign prostate hyperplasia and prostate cancer. Although no single study has yet demonstrated statistically significant results for the use of iodine for the treatment of prostate cancer, multiple studies of iodine indicate a trend that links moderate iodine consumption with a lower risk factor for prostate cancer (Key T J A, Silcocks P B, Davey G K, Appleby P N, and Bishop D T “A case-control study of diet and prostate cancer”, British Journal of Cancer (1997) 76(5), 678-687 and Hoption Cann S A, Qiu Z, and van Netten C “A Prospective Study of Iodine Status, Thyroid Function, and Prostate Cancer Risk: Follow-up of the First National Health and Nutrition Examination Survey” NUTRITION AND CANCER (2007) 58(1): 28-34). However, consumption of supraphysiologic levels of iodine, i.e., above 1.1 mg per day, is not common in many countries where these studies have been performed. Such levels of iodine consumption are required in order to have sufficient iodine available to provide prophylactic benefits for prostate health and to provide a significantly lower risk of prostate cancer.

Further evidence of the effect of iodine for maintaining prostate health comes from epidemiological studies of Japanese populations in comparison to western populations. The typical Japanese diet includes 25 times higher levels of total iodine consumption in comparison to a typical diet in western countries. According to Hoption Cann et al., Japanese people also have some of the lowest breast and prostate cancer rates in the world and this distinction is lost by immigrants to the United States and successive generations, indicating that the effect is not solely genetic. (Hoption Cann S A, Qiu Z, and van Netten C “A Prospective Study of Iodine Status, Thyroid Function, and Prostate Cancer Risk: Follow-up of the First National Health and Nutrition Examination Survey” NUTRITION AND CANCER (2007) 58(1): 28-34). Olvera-Caltzontzin et al. state that prostate cancer rates are approximately 3.7 times higher in the United States than in Japan (Olvera-Caltzontzin P, Delgado G, Aceves C, and Anguiano B “Iodine uptake and Prostate Cancer in the TRAMP Mouse Model” Molecular Medicine (2013) 19:409-416).

In addition epidemiological studies and studies of low and moderate consumption of iodine, additional evidence of the effectiveness of iodine for the treatment of prostate conditions or disease is offered by studies in in vitro and animal studies, which show uptake of molecular iodine into prostate cells and inducement of apoptosis in cancer and normal prostate cell lines.

Iodine supplementation has been suggested for treating fibrocystic breast condition and benign prostate hyperplasia, among other conditions. The use of iodine is complicated by its administration in many different forms. Iodine may be delivered as organically-bound iodine (e.g., caseinated iodine), inorganic iodine, and molecular iodine, i.e., I₂. Each of these forms of iodine has been used to treat conditions other than fibrocystic breast condition such as iodine deficiency, goiter, hyperthyroidism, and others. Some ingestible iodine oral dosage forms include iodide salts having no mechanism for efficient conversion of iodide to molecular iodine prior to absorption into internal tissue. Other oral dosage forms include an unstable form of molecular iodine that reacts to form iodide salts prior to significant interaction with internal tissue. Examples of iodide salts include potassium iodide and sodium iodide, both of which are used in iodized salt in many countries. Iodide for example potassium iodide (CAS Registry Number: 7681-11-0), iodate for example potassium iodate (CAS Registry Number: 7758-05-6), and molecular iodine (CAS Registry Number: 7553-56-2) differ significantly in their chemical nature and physiologic interactions with the human body. Iodide (typically administered with a suitable counter-ion) has an anionic charge and is hydrophilic, stable in water, and highly water soluble. In contrast, molecular iodine is uncharged and hydrophobic, and reacts with water at pH values above approximately 6. Molecular iodine also has a significant vapor pressure and sublimes at room temperature. These characteristics are incompatible with the use of molecular iodine in many solid and liquid dosage forms.

Significant differences also exist between iodide and molecular iodine in terms of the toxicity and therapeutic efficacy of orally administered forms thereof (K. D. Thrall, Ph.D. dissertation, Washington State University, Program in Pharmacology and Toxicology, December 1990). Work by Aceves et al. demonstrates that although there is a small uptake difference between molecular iodine and iodide in the breast there is a 3-4 fold difference in uptake in the thyroid (Aceves C, Anguiano B, and Del G. “The Extrathyronine Actions of Iodine as Antioxidant, Apoptotic, and Differentiation Factor in Various Tissues” Thyroid (2013) 23(8): 938-946). This means that higher levels of molecular iodine can be absorbed without thyrotoxic effects. Despite the differences in pharmacologic and toxicological activity between molecular iodine and iodide as noted supra, the term “iodine” is frequently used interchangeably to refer to these two species, as well as to several other distinct chemical species that contain iodine atoms. Iodide salts are frequently simply described as iodine due to the fact that the iodide salts contain ionic forms of atomic iodine. Much of the prior art, including the scientific medical literature, uses the term “iodine” somewhat imprecisely in this regard.

Despite the benefits of molecular iodine as an active ingredient in comparison to iodide and iodate based dosage forms, the instability of molecular iodine in many oral dosage forms limits its use in practice. To overcome the instability of molecular iodine in a liquid or solid oral dosage form, some dosage forms choose to bind molecular iodine to another material such that the molecular iodine is released in the stomach or to deliver components that react to form molecular iodine in the stomach. However, the stomach may contain food, drugs, and other materials that can interact with the dosage form to limit the release or formation of molecular iodine in the stomach.

In a Phase II clinical study, dosing with a molecular iodine-based dosage form as described in U.S. Pat. Nos. 5,885,592 and 6,248,335 was demonstrated as a safe and effective treatment for treating symptoms related to fibrocystic breast condition. However, some physicians and patients have concerns regarding consumption of supraphysiologic levels of iodide, which are contained in some iodine supplements. For example, some believe that high levels of iodide is potentially unsafe due to risks of iodide being absorbed by the thyroid and disrupting the thyroid hormone chemistry for patients who have underlying thyroid conditions that prevent their thyroids from adapting to high levels of iodide. Others believe that the levels of iodate delivered by some iodine dosage forms may pose safety risks despite the use of iodate as an additive in salt, bread, and other common processed foods. Bürgi, et al. state “Because it is more stable than iodide, most health authorities preferentially recommend iodate as an additive to salt for correcting iodine deficiency. Even though this results in low exposure of at most 1700 μg/d, doubts have recently been raised whether the safety of iodate has been adequately documented.” High levels of iodate are highly toxic to the eye and can lead to blindness (Bürgi, et al. “The Toxicology of Iodate: A Review of the Literature” Thyroid 2001; 11(5); 449-456).

To deliver a consistent dose of molecular iodine to the stomach and to address potential concerns related to high levels of iodide and iodate, there remains a need for a dosage form and related therapies for treatment of conditions such as fibrocystic breast condition and breast cancer that can deliver supraphysiologic doses of molecular iodine in an amount effective to result in a notable reduction in one or more symptoms associated with conditions such as fibrocystic breast condition or breast cancer, that deliver a controlled amount of molecular iodine, and/or that limit the amounts of iodide and iodate that are available in the stomach.

### BRIEF SUMMARY

The present disclosure overcomes one or more of the limitations of the prior art by providing, in one aspect, an oral dosage form that comprises a source of iodine and a reactive agent, wherein (i) the total iodine in the oral dosage form is 3 to 60 milligrams, (ii) the source of iodine reacts with the reactive agent in the stomach or simulated gastric fluid to form molecular iodine, and (iii) the amount of the source of iodine or the amount of the reactive agent is in excess of the amount needed for the reaction to form molecular iodine, i.e., one of the source of iodine or the reactive agent is present in excess of the stoichiometric amount required for the reaction.

In some embodiments, the oral dosage form further comprises a source of carboxylate or phosphate. For example, in some embodiments, the oral dosage form further comprises a source of carboxylate. In some embodiments, the oral dosage form further comprises a source of phosphate.

In some embodiments, the oral dosage form further comprises a catalyst that increases the rate of the reaction between the source of iodine and the reactive agent in (ii).

In one or more further embodiments, the oral dosage form further comprises a scavenger that reacts with at least a portion of the excess in (iii). In one or more related embodiments, the scavenger is a protein comprising a sulfhydryl (i.e., thiol) group. In one or more particular embodiments, the scavenger is a protein comprising a cysteine or a methionine. In some embodiments, the scavenger is a thiol-containing small molecule. In one or more additional embodiments, the scavenger is cysteine or glutathione.

In yet some further embodiments related to the oral dosage form, the rate of reaction between the source of iodine and the reactive agent is faster than the rate of reaction between the excess amount of the source of iodine or the reactive agent and the scavenger.

In some embodiments, the dosage form is an extended-release dosage form. In some embodiments, the dosage form releases no more than 5% or no more than 25% of the source of iodine or the reactive agent in the first 20 minutes when tested according to U.S. Pharmacopeia <711>.

In some embodiments, the dosage form is a delayed-release dosage form. In some further embodiments, more than 10% of an active ingredient of the dosage form remains undissolved after 2 hours in a test according to Method A for delayed-release dosage forms in U.S. Pharmacopeia <711>. In some further embodiments, more than 10% of a scavenger of the dosage form remains undissolved after 2 hours in a test according to Method A for delayed-release dosage forms in U.S. Pharmacopeia <711>. In some further embodiments, more than 10% of a catalyst of the dosage form remains undissolved after 2 hours in a test according to Method A for delayed-release dosage forms in U.S. Pharmacopeia <711>.

In some embodiments, the scavenger has a pH dependent rate of reaction such that less than 30% of the reaction is completed after a two-hour acid stage as defined in U.S. Pharmacopeia <711> Method A for delayed-release dosage forms and the rate of the reaction increases when subsequently placed in the buffer stage of that method. In some embodiments, less than 10% of this reaction is completed. In some embodiments, less than 5% of this reaction is completed.

In some embodiments, the reaction between the source of iodine and the reactive agent in (ii) is an oxidation-reduction reaction.

In some embodiments, the molar ratio of the source of iodine to the reactive agent in the oral dosage form exceeds the corresponding molar ratio for the reaction(s) to form molecular iodine. In some embodiments, the molar ratio of the source of iodine to the reactive agent in the oral dosage form exceeds the corresponding molar ratio for the reaction(s) to form molecular iodine by at least 25%, preferably by an amount in the range of 25% to 1000%, more preferably by an amount in the range of 50% to 500%, and most preferably by an amount in the range of 50% to 300%.

In some embodiments, the molar ratio of the reactive agent to the source of iodine in the oral dosage form exceeds the corresponding molar ratio for the reaction(s) to form molecular iodine. In some embodiments, the molar ratio of the reactive agent to the source of iodine in the oral dosage form exceeds the corresponding molar ratio for the reaction(s) to form molecular iodine by at least 25%, preferably by an amount in the range of 25% to 1000%, more preferably by an amount in the range of 50% to 500%, and most preferably by an amount in the range of 50% to 300%.

In some embodiments of the oral dosage form, the source of iodine and the reactive agent are mixed in a homogeneous distribution.

In one or more particular embodiments, the source of iodine in the oral dosage form comprises iodide and the reactive agent comprises iodate.

In some embodiments, the molar ratio between the iodide and the iodate in the dosage form is in a range of 6.5:1 to 100:1. In some other embodiments, the molar ratio between the iodide and the iodate in the dosage form is in a range of 1:100 to 4:1, in a range of 1:50 to 3:1, or in a range of 10:1 to 50:1.

In some embodiments, the molar ratio between the iodate and the iodide in the dosage form is in a range of 1:4 to 5:1. In some other embodiments, the molar ratio between the iodide and the iodate in the dosage form is in a range of 3:10 to 5:1, in a range of 1:2 to 5:1, or in a range of 1:4 to 1:1.

In some embodiments of the oral dosage form, the source of iodine comprises iodide, and the reactive agent is selected from the group consisting of an iodate salt, hydrogen peroxide, a source of iodate, and a source of hydrogen peroxide.

In yet some additional embodiments, the oral dosage form further comprises a pH control agent such that the effective pH of the oral dosage form is between 7.0 and 12.0.

In yet some additional embodiments, the oral dosage from further comprises a pH buffer agent that adjusts the pH of 0.5 liter of simulated gastric fluid to a pH in the range of 4.0-8.0, or in the range of 4.0-6.0.

In yet some additional embodiments, the oral dosage from further comprises at least 300 mg to 1000 mg of a pH buffer agent.

In yet some additional embodiments, the oral dosage from further comprises an absorption matrix that stabilizes molecular iodine in solution.

In yet some further embodiments of the oral dosage form, the source of iodine comprises iodide and the reactive agent is a ferric salt.

In certain embodiments, the total iodine in the oral dosage form is 6 to 50 milligrams.

In certain embodiments, the oral dosage form further comprises a lipid excipient. In some embodiments, the lipid excipient comprises a medium chain triglyceride or a long chain triglyceride.

In yet another aspect, provided herein is a method of treatment or prophylaxis of a condition or disease responsive to treatment with iodine, comprising the steps of (i) providing an oral dosage form as described herein at least once daily to a human patient for a period of at least 30 days. In some aspects, the condition or disease is fibrocystic breast condition. In some aspects, the condition or disease is breast cancer.

In yet an additional aspect, provided herein is a method for fabricating an oral dosage form comprising the steps of (i) providing a source of iodine, (ii) providing a reactive agent, and (iii) combining the source of iodine and the reactive agent to form an oral dosage form with an effective pH above 7, wherein either the source of iodine or the reactive agent is provided in excess of the corresponding molar ratio for the reaction(s) to form molecular iodine.

Additional embodiments of the oral dosage form, related methods, components of the oral dosage form, and the like will be apparent from the following description, examples, figures and claims. These and other objects and features of the disclosure will become more fully apparent when read in conjunction with the following detailed description.

### BRIEF DESCRIPTION OF THE DRAWINGS

FIG. 1 is a photograph showing the oil-water mixtures produced by an experiment in which potassium iodate and potassium iodide were mixed in oil-water mixture as described in Example 2. In this mixture, the water phase simulates the pH of the stomach. Molecular iodine is hydrophobic and partitions into the oil phase. Other iodine species, such as iodide and triiodide, partition into the water phase.

FIG. 2 is a graph indicating the absorbance of hydrophilic and hydrophobic iodine species within an oil-water mixture as described in Example 2.

FIG. 3 is a graph indicating the cell viability 24 hours after adding cells to each of the seven cell media mixtures CM1 to CM7 normalized to the cell viability 24 hours after adding cells from the same cell line to cell media alone (i.e., CM1) as described in Example 3.

### DETAILED DESCRIPTION

The present disclosure overcomes one or more of the problems associated with an oral dosage form comprising iodine. In one aspect, provided herein is a stable oral dosage form that delivers a more consistent dose of molecular iodine to a patient by providing an excess of one of at least two components that react together to form molecular iodine in the stomach. This reduces the variability in the amount of molecular iodine that is formed in the stomach for fed and fasted conditions.

The present invention will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As can be appreciated from the foregoing and following description, each and every feature described herein, and each and every combination of two or more of such features, is included within the scope of the present disclosure provided that the features included in that such combinations are not inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

All publications, patents and patent applications cited herein are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document, which are incorporated herein by reference.

### Definitions

It must be noted that, as used in this specification, the singular forms “a,” “an,” and “the” include plural referents unless the context clearly dictates otherwise. Thus, for example, reference to an “active ingredient” includes a single ingredient as well as two or more different ingredients, reference to a “scavenger” includes a single scavenger as well as to two or more different scavengers, reference to a “catalyst” includes a single catalyst as well as two or more different catalysts, and the like.

In describing and claiming the present invention, the following terminology will be used in accordance with the definitions described below.

The term “oral dosage form” refers to a dosage form that comprises pharmaceutically acceptable ingredients and is to be administered orally to an animal or human. The oral dosage form may be classified, for example, as a dietary supplement, a drug, and/or a biologic material depending on the specific contents of the oral dosage form, its intended use, and the country in which it is sold. A dosage form is intended to treat or prevent a condition or disease, or the symptoms associated therewith. It is also a means of addressing a lack of availability of a critical material in an individual's diet. It is not a requirement that a dosage form is classified as a drug. It may, for example, be a dietary supplement if it is intended to treat or prevent a condition.

The term “molecular iodine” refers to diatomic iodine, which is represented by the chemical symbol I₂ (CAS Registry Number: 7553-56-2) whether in the liquid or solid state. The concentration of molecular iodine can be measured by the method described by Gottardi (Gottardi, W., Fresenius Z. Anal. Chem. Vol. 314, pp. 582-585, 1983).

The term “iodide” or “iodide anion” refers to the species that is represented by the chemical symbol I⁻ (CAS Registry Number: 20461-54-5). Suitable counter-ions for the iodide anion include sodium, potassium, calcium, and the like.

The term “iodine” is used to refer to the element iodine in any form. Similarly, the term “source of iodine” refers to an entity that contains at least one iodine atom. Examples of sources of iodine include iodide anion per se, salts of iodide (e.g., potassium iodide, sodium iodide, and calcium iodide), molecular iodine, triiodide (I₃⁻), organically complexed forms of iodine, covalently bound forms of iodine, iodate, and polyiodides.

The term “total iodine” in a sample refers to the amount of iodine, irrespective of form, from all iodine containing components within a sample. The concentration of total iodine can be measured by a thiosulfate titration as described in the United States Pharmacopeia (USP).

The term “ratio of molecular iodine to total iodine” in a sample refers to the ratio of the concentration by weight of iodine in all molecular iodine (I₂) in the sample divided by the concentration by weight of total iodine from all iodine containing components within the sample. The concentration of molecular iodine can be measured by a thiosulfate titration as described in the United States Pharmacopeia (USP).

The term “supraphysiologic” in relation to a chronic dosing of iodine refers to doses exceeding 1.1 mg per day of total iodine.

The term “simulated gastric fluid” (SGF) refers to a solution formed by dissolving 2.0 g of sodium chloride in 7.0 ml of hydrochloric acid and sufficient water to produce 1000 ml of solution. This solution has a pH of approximately 1.2.

The term “thiol” refers to an organosulfur compound that contains a carbon-bonded sulfhydryl group. Examples of thiols include cysteine and glutathione.

The term “scavenger” refers to a substance in an oral dosage form that reacts with or otherwise deactivates impurities and/or unwanted excess reactive substances in the dosage form. For purposes of this application, a scavenger may also be used to react with a component of a beneficial reactant in a desired reaction when the reactant is provided in excess of the amount needed for the reaction. As an example, if iodide reacts with iodate in a molar ratio of 5:1 and a molar ratio of 2:1 is provided, a scavenger may be used to react with excess iodate to limit the interaction of iodate with the body or with other substances that have been consumed, such as food, vitamins, or drugs. Preferably, the scavenger should not react readily with molecular iodine.

The term “catalyst” refers to a substance that increases the rate of a reaction that leads to an increase in the rate of formation of molecular iodine. For clarity, a catalyst for a reaction between an iodide salt and an iodate salt, for example, would include not only a direct reaction between these two reactants in the formation of molecular iodine, but also the acceleration of one or more intermediate reactions such that the rate of creation of molecular iodine is increased.

The term “absorption matrix” is used to describe a material that absorbs molecular iodine without forming covalent bonds with the molecular iodine.

The term “enteric coating” refers to a substance that forms a delayed-release dosage form for the ingredients mixed therein. An oral dosage form can be tested to determine whether its ingredients are coated by an enteric coating by following the procedures described by Method A for delayed-release dosage forms in U.S. Pharmacopeia <711> and testing the amount of the active ingredients released.

The term “delayed-release dosage form” refers to an oral dosage form in which more than 10% of an active ingredient, a scavenger, or a catalyst remains undissolved after 2 hours in a test according to Method A for delayed-release dosage forms in U.S. Pharmacopeia <711>.

The term “extended release dosage form” refers to an oral dosage form in which less than 10% of an active ingredient, a scavenger, or a catalyst is released in the first 20 minutes in a test according to the method for extended-release dosage forms in U.S. Pharmacopeia <711>.

The term “pharmaceutically acceptable” in reference to a formulation component or ingredient is one that may be included in the oral dosage forms/dosage forms provided herein and that causes no significant adverse toxicological effects in the patient at specified levels, or if levels are not specified, at levels known to be acceptable by those skilled in the art. All ingredients in the oral dosage forms described in this application are pharmaceutically acceptable. For clarity, molecular iodine may cause one or more side effects and inclusion of the source of iodine and a reactive agent that react together to form molecular iodine with a side effect profile that is acceptable from a regulatory perspective for such ingredients will be deemed to be “pharmaceutically acceptable” levels of those ingredients.

The terms “effective amount” or “pharmaceutically effective amount” or “therapeutically effective amount” of an oral dosage form or oral dosage form as provided herein, refers to a non-toxic but sufficient amount of the oral dosage form or dosage form or ingredient therein to provide the desired response, such as preventing, diminishing, or eliminating one or more symptoms of fibrocystic breast condition or of breast cancer in a subject. The exact amount required will vary from subject to subject, depending on the species, age, and general condition of the subject, the severity of the condition being treated (e.g., fibrocystic breast condition or breast cancer), the particulars of the dosage form employed, and the like. An appropriate “effective” amount in any individual case may be determined by one of ordinary skill in the art using routine experimentation.

“Treatment” or “treating” as used herein refers to controlling, preventing or otherwise reducing the occurrence, severity, or relapse of an identified symptom, condition or disease in individuals afflicted with or prone to develop such symptoms, condition or disease.

The “pH” or “effective pH” of an oral dosage form or dried granulation is measured by preparing a 10% (w/v) solution of the oral dosage form or granulation in distilled water and determining the pH.

The term “pH control agent” refers to chemical(s) that control the effective pH of a dried granulation for an oral dosage form. Suitable pH control agents include sodium carbonate, calcium carbonate, potassium carbonate, magnesium carbonate, sodium hydroxide, bentonite, dibasic calcium phosphate dehydrate, magnesium oxide, magnesium trisilicate, sodium bicarbonate, dibasic sodium phosphate, and tribasic potassium phosphate. The level of pH control agent is typically deliberately too low to significantly impact the pH of the stomach.

The term “pH buffer agent” refers to chemical(s) that control the effective pH of 0.5 liters of SGF. This pH measurement in SGF is designed to approximate the effect of the pH buffer agent on the pH of the stomach. Suitable pH buffer agents include sodium carbonate, calcium carbonate, potassium carbonate, magnesium carbonate, sodium hydroxide, bentonite, dibasic calcium phosphate dehydrate, magnesium oxide, magnesium trisilicate, sodium bicarbonate, dibasic sodium phosphate, and tribasic potassium phosphate.

A mixture that is referred to, interchangeably, as “homogeneous” or as having a “homogeneous distribution” is a mixture in which the concentration for each of the sampled ingredients typically has a relative standard deviation of less than 5% when 10 or more samples of 0.1 to 10 mg are taken from different locations within the mixture where a source of iodine is present. For an oral dosage form, the homogeneous distribution is usually assessed for the dried granulation matrix that contains the active ingredients prior to compression forming a solid oral dosage form. If an oral dosage form includes multiple granulation mixtures, such as a granulation mixture with an enteric coating and a granulation mixture without an enteric coating, whether the oral dosage form is homogenous will be determined based on the homogenous distribution of each granulation mixture separately. It is not required that the two mixtures be evenly distributed within an oral dosage form for it to be considered a homogenous distribution of selected ingredients. If an oral dosage form includes multiple portions, such as a portion with an enteric coating and a portion without an enteric coating, then the source of iodine and the reactive agent are said to be mixed in a homogenous distribution if they are mixed in a homogenous distribution within either portion of the solid oral distribution.

The terms oxidant, oxidizer, and oxidizing agent are used interchangeably to refer to an element or compound that accepts one or more electrons in an oxidation-reduction reaction from at least one other element, compound, molecule or chemical species.

The terms reductant, reducer, and reducing agent are used interchangeably to refer to an element or compound that donates one or more electrons in an oxidation-reduction reaction to at least one other element, compound, molecule or chemical species.

A drug is said to be “stabilized” by a material in an oral dosage form if an oral dosage form comprising all of the same materials in the same proportions, but with the material removed would lose potency more quickly than the original oral dosage form when stored at 25° C. and 60% relative humidity in a dark environment. For clarity, when performing the replacement, the percentage of the drug is not increased, but instead the removed material is effectively replaced by equivalent proportions from the rest of the oral dosage form excluding the drug.

The abbreviation “(w/w)” indicates that relative concentrations presented on a “weight for weight” basis (i.e., percentages refer to a percentage of the total weight), rather than on the basis of volume.

The abbreviation “(w/v)” indicates that relative concentrations presented on a “weight for volume” basis, wherein 1% corresponds to 1 gram per 100 milliliters of solution.

The abbreviation “mg” shall be used interchangeably with the term “milligram(s).”

The abbreviation “mL” shall be used interchangeably with the term “milliliter(s).”

The abbreviation “° C.” shall be used interchangeably with the term “degrees Celsius.”

The term “pharmaceutically acceptable” in reference to an entity or ingredient is one that may be included in the oral dosage forms provided herein and that causes no significant adverse toxicological effects in the patient at specified levels, or if levels are not specified, in levels known to be acceptable by those skilled in the art. All ingredients in the oral dosage forms described herein are provided at levels that are pharmaceutically acceptable. For clarity, active ingredients may cause one or more side effects and inclusion of the ingredients with a side effect profile that is acceptable from a regulatory perspective for such ingredients will be deemed to be “pharmaceutically acceptable” levels of those ingredients.

“Pharmaceutically acceptable salt” denotes a salt form of a drug or active ingredient having at least one group suitable for salt formation that causes no significant adverse toxicological effects to the patient. Reference to an active ingredient as provided herein is meant to encompass its pharmaceutically acceptable salts, as well as solvates and hydrates thereof. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitable pharmaceutically acceptable salt forms and methods for identifying such salts are found in, e.g., Handbook of Pharmaceutical Salts: Properties, Selection and Use, Weinheim/Zürich: Wiley-VCH/VHCA, 2002; P. H. Stahl and C. G. Wermuth, Eds.

“Therapeutically effective amount” is used herein to mean the amount of a pharmaceutical preparation, or amount of an active ingredient in the pharmaceutical preparation, that is needed to provide a desired level of active ingredient in the bloodstream or in a target tissue. The precise amount will depend upon numerous factors, e.g., the particular active ingredient, the components and physical characteristics of the pharmaceutical preparation, intended patient population, patient considerations, and the like, and can readily be determined by one skilled in the art, based upon the information provided herein and available in the relevant literature.

The term “patient” refers to a living organism suffering from or prone to a condition that can be prevented or treated by administration of an oral dosage form as provided herein, and includes both humans and animals.

In many cases, the patent application describes ranges of values. Such ranges shall be construed to include the endpoints of the range unless doing so would be inconsistent with the text or otherwise noted.

### Oral Dosage Form

Oral dosage forms have been previously described that combine precise amounts of iodide and iodate salts to form molecular iodine in the stomach (U.S. Pat. Nos. 5,885,592 and 6,248,335). However, as recognized by the Applicants, the rate of reaction for such combinations drops precipitously as the components of the reaction are consumed, thus preventing the reaction from efficiently proceeding to completion and thus potentially leaving undesirable iodide or iodate to be absorbed. This limits the amount of molecular iodine that is formed. Additionally, the amount of molecular iodine that is formed in the stomach can vary significantly depending on the amount of reactive material, such as food, that is in the stomach.

The reaction between iodate and iodide salts, also known as the Dushman reaction, is highly complex with many complex equilibria and intermediates. Schmitz elucidates the steps of the reaction (Schmitz G “Kinetics and mechanism of the iodate-iodide reaction and other related reactions” Phys. Chem. Chem. Phys. (1999)1:1909-1914). The stoichiometry is widely variable, with an 8:1 molar ratio being preferred in some embodiments since molecular iodine is rapidly converted to triiodide. In embodiments an optimal formulation is provided herein to ensure that the correct dosage of molecular iodine is delivered following ingestion. Since triiodide is more stable than molecular iodine for physiologic pH ranges, triiodide represents a stable form of molecular iodine for delivery to tissues such as breast and prostate at higher levels while not delivering a proportionate increase to tissues such as thyroid that rely primarily on the sodium iodide symporter to transport iodide into the cells. Since the reaction in the stomach will be competing with a range of side reactions, there exists a need to formulate an oral dosage product to maximize the kinetics of the desired reaction. This can be accomplished for example, by the following four methodologies, individually or in combinations thereof.

First, one or more catalysts that promote a reaction between an iodate and an iodide salt to form molecular iodine can be incorporated into the dosage form. These include, for example, phosphate salts, carboxylate salts & bromide salts. For example, a phosphate salt or a carboxylate salt catalyzes the Dushman reaction to increase the rate of reaction.

Second, the pH of the stomach (as simulated by 0.5 liters of SGF) can be increased by the dosage form to the range of 4.0 to 8.0. This can be achieved, for example, by incorporating into the oral dosage form a suitable amount of an antacid such as calcium carbonate, magnesium carbonate, magnesium hydroxide, magnesium bicarbonate, or combinations thereof. Adjustment of the pH is effective to diminish the ready conversion of potassium iodate (a weak acid) in the stomach to periodic acid. The conversion of potassium iodate to periodic acid, if unchecked, will slow the rate of, for example, the Dushman reaction significantly. This effect can be simulated by the use of simulated gastric fluid.

Third, molecular iodine can be isolated after its formation so that it is not reactive by preferential absorption into an absorption matrix. Examples of such absorption matrices include alpha, beta and gamma cyclodextrin (CD), low molecular weight polyvinyl pyrrolidone (PVP), cellulose, lignin, hydroxypropyl cyclodextrin, and polyanionic beta-cyclodextrin derivative with a sodium sulfonate salt separated from the lipophilic cavity by a butyl ether spacer group, or sulfobutyl ether (SBE) (e.g., CAPTISOL®).

Fourth, an excess of one of the compounds of the reaction that forms molecular iodine can be provided. For example, in the Dushman reaction, the provided molar ratio of iodide to iodate can be lower than the 5:1 ratio specified by the Dushman reaction, to less than 4:1. Since this lower molar ratio will leave an excess of iodate in the stomach, in preferred embodiments, the incorporation of an iodate scavenger in the oral dosage form is preferred. Examples of such scavengers include any molecule or material containing a sulfhydryl group. Further examples include glutathione, cysteine, and ergothioneine without limitation. These scavenger compounds, and scavenger compounds such as these, are preferred because they do not readily react with molecular iodine, and do not react readily with iodate at low pH such as found in the stomach. Without being bound by theory, it is expected that these thiol-containing compounds will react with iodate in the neutral pH of the small intestine and will not compete with the iodine-forming reactants in the stomach.

Generally, an oral dosage form and related methods for making the oral dosage form are provided herein. The oral dosage form generally comprises a source of iodine and a reactive agent, wherein either the source of iodine or the reactive agent is provided in excess of the amount needed for the reaction to form molecular iodine, i.e., one of the source of iodine or the reactive agent is present in excess of the stoichiometric amount required for the reaction to form molecular iodine.

Illustrative examples of the source of iodine are iodide salts (e.g. potassium iodide, sodium iodide), iodate salts (e.g. potassium iodate, sodium iodate), molecular iodine, and combinations thereof. Any one of the foregoing is suitable for use in the instant oral dosage form, either singly, or in combination.

Illustrative examples of a reactive agent include oxidants, iodate salts (e.g. potassium iodate, sodium iodate), sources of ferric iron or Fe(III) (e.g., ferric oxide), and reductants (e.g., sources of hydrogen peroxide or peroxidases). Examples of reactive agents further include sources of reactive agents, such as enzymatic sources of hydrogen peroxide (e.g., glucose oxidase).

Molecular iodine itself is generally not stable when comprised in solid oral dosage forms under normal storage conditions. Therefore, it is preferable to incorporate into the instant composition ingredients that generate molecular iodine in the stomach or in simulated conditions thereof, such as in SGF (simulated gastric fluid), due both to the reduced toxicity of molecular iodine in comparison to iodide, and the ability to safely administer to a subject much higher levels of molecular iodine than have been previously considered to be both acceptable and safe. Additional details can be found in co-pending U.S. Patent Publication No. 2015/0147400, which is incorporated herein by reference. The in vivo or in vitro generation of molecular iodine can be accomplished by combining a source of iodine with a reactive agent, where the desired ultimate application is an in vivo reaction in the stomach. SGF is used as a model system to indicate whether a reaction between the source of iodine and the reactive agent will generate molecular iodine in the acidic environment of the stomach. Exemplary combinations that can be used to generate molecular iodine in SGF include the following categories: (1) oxidants comprising iodine (iodine oxidizer) combined with reductants comprising iodine (iodine reducer), (2) sources of ferric iron (e.g., ferric salts) plus sources of iodide (e.g., iodide salts), (3) sources of iodide plus oxidants, and (4) sources of iodate plus reductants. Preferably, ingredients of the solid oral dosage form generate molecular iodine when mixed with SGF (and in stomach acid) and are present in the solid dosage form to provide a total iodine content therein of from about 3 to 60 mg (e.g., 3, 5, 10, 20, or 60 mg), about 6 to 50 mg (e.g., 6, 15, 20, or 50 mg), or about 9 to 40 mg (e.g., 9, 16, 32, or 40 mg). The total iodine content in the solid dosage form may be, for example, from greater than about 6 mg to 60 mg, from greater than about 6 mg to 50 mg, from greater than about 6 mg to 40 mg, from about 7 mg to 50 mg, from about 8 mg to 40 mg, from about 9 mg to 30 mg, from about 9 mg to 25 mg, or from about 10 mg to 25 mg. In some embodiments, the total iodine content in the solid dosage from may be, for example, from about 3-5 mg, 3-6 mg, 3-7 mg, 3-8 mg, 3-9 mg, 3-10 mg, 3-15 mg, 3-20 mg, 3-30 mg, 3-40 mg, 3-50 mg, 5-6 mg, 5-7 mg, 5-8 mg, 5-9 mg, 5-10 mg, 5-15 mg, 5-20 mg, 5-30 mg, 5-40 mg, 5-50 mg, 5-60 mg, 6-7 mg, 6-8 mg, 6-9 mg, 6-10 mg, 6-15 mg, 6-20 mg, 6-30 mg, 6-40 mg, 6-50 mg, 6-60 mg, 7-8 mg, 7-9 mg, 7-10 mg, 7-15 mg, 7-20 mg, 7-30 mg, 7-40 mg, 7-50 mg, 7-60 mg, 8-9 mg, 8-10 mg, 8-15 mg, 8-20 mg, 8-30 mg, 8-40 mg, 8-50 mg, 8-60 mg, 9-10 mg, 9-15 mg, 9-20 mg, 9-30 mg, 9-40 mg, 9-50 mg, 9-60 mg, 10-15 mg, 10-20 mg, 10-30 mg, 10-40 mg, 10-50 mg, 10-60 mg, 15-20 mg, 15-30 mg, 15-40 mg, 15-50 mg, 15-60 mg, 20-30 mg, 20-40 mg, 20-50 mg, 20-60 mg, 30-40 mg, 30-50 mg, 30-60 mg, 40-50 mg, 40-60 mg, or about, 50-60 mg. It is preferred that the distribution of the source of iodine and the reactive agent in the dosage form be homogenous to enable effective mixing between the source of iodine and the reactive agent.

Examples of combinations falling within category 1 supra are described in U.S. Pat. Nos. 5,885,592 and 6,248,335, which are herein incorporated by reference. For this category, the ratio of the iodine oxidizer to iodine reducer is selected so that the predominant species of iodine in the stomach (as simulated by SGF) is molecular iodine. For salts of iodide and iodate, the molar ratio for the reaction between the iodide and iodate is 5:1. For combinations in category 1, either iodide or iodate may serve the role of the source of iodine while the other serves the role of the reactive agent to thereby generate molecular iodine.

For many of the ferric (Fe (III)) salts described in category 2, the iron is converted from ferric to ferrous iron (Fe (II)) following the oxidation of iodide to molecular iodine. Examples in category 2 include combinations of ferric oxide and potassium iodide and/or sodium iodide. When these combinations are placed in a low pH environment, such as stomach acid or SGF, they undergo the following reaction(s): Fe₂O₃+2H₃O⁺+2KI+2Cl⁻→2FeO+I₂+3 H₂O+2KCl and/or Fe₂O₃+2H₃O⁺+2NaI+2Cl⁻→2FeO+I₂+3 H₂O+2NaCl. In this example, the molar ratio for the reaction between the source of iodine (i.e., potassium iodide or sodium iodide) and the reactive agent (i.e., ferric oxide) is 2:1.

Another suitable combination is an iodide salt plus a nitrate salt. When this combination of salts is placed in a low pH environment, such as stomach acid or SGF, the following reaction takes place: NO₃⁻+4H⁺+2I⁻→NO+I₂+2H₂O, via an intermediate reaction that produces HNO₂ and H₂O. In this example, the molar ratio for the reaction between the source of iodine (i.e., an iodide salt) and the reactive agent (i.e., a nitrate salt) is 2:1. Another suitable combination falling in this category include an iodide salt plus a source of hydrogen peroxide or an organic hydroperoxide, in combination with a peroxidase as a catalyst. Examples of organic hydroperoxides include, e.g., ethyl hydroperoxide, propyl hydroperoxide, 1-(cyclopropylmethyl)propyl hydroperoxide, 1,1-dimethyl-3(2-methylphenyl)propyl hydroperoxide, and the like. An organic hydroperoxide is generally defined as R—O—O—H, where R is generally a C1-C10 alkyl, aralkyl or cycloalkyl group. Examples of peroxidases include horseradish peroxidase, soybean peroxidase, lactoperoxidase, myerloperoxidase, NADH peroxidase, NDAPH peroxidase, fatty-acid peroxidase, cytochromes-c peroxidase, catalase, glutathione peroxidase, L-ascorbate peroxidase, phospholipid-hydroperoxide glutathione peroxidase, manganese peroxidase, lignin peroxidase, peroxiredoxin, versatile peroxidase, glutathione amide-dependent peroxidase, bromide peroxidase, dye decolorizing peroxidase, prostamide/prostaglandin F2α synthase, catalase-peroxidase, and hydroperoxy fatty acid reductase.

In water, molecular iodine reacts rapidly in water with iodide to form a triiodide and/or polyiodide anion and reaches an equilibrium based on the pH of the solution. For purposes of calculating the molar ratio for reactants for a reaction to form molecular iodine in this application, it is assumed that no triiodide anions are formed. So, for example, in a reaction between iodide and iodate, the molar ratio for reactants is 5:1 because this is the molar ratio of iodide and iodate ions needed to form molecular iodine (without forming triiodide).

A component (i.e., a source of iodine or a reactive agent) is described as an “excess component” if it is provided in excess of that needed for the reaction(s) to make molecular iodine. Since the rate of a reaction is proportional to the product of the concentrations of the reactants in a solution, the rate of reaction between the source of iodine and the reactive agent proceeds more rapidly towards one hundred percent completion if one of the two components is provided in excess of the stoichiometric amount required for the reaction to make molecular iodine. This is particularly true as the reaction nears completion. Absent an excess component, the rate of reaction slows exponentially as the reactants are consumed. By providing an excess of one of the reactants, the rate reduction can, in some embodiments, be maintained and preferably never drops below that of the corresponding reaction(s) carried out without an excess component. By thus increasing the rate of reaction, i.e., the production of molecular iodine, the source of iodine and reactive agent have less time to react and/or interact with other materials that may be present in the stomach via side reactions, such as food that has been ingested, thereby resulting in less variability in the amount of iodine that is produced. Altering the dosage form to include an excess of either the source of molecular iodine or the reactive agent may also diminish the occurrence of adverse side effects, or decrease the rate of occurrence of side effects, and/or reduce the occurrence of drug interactions, particularly for a component that is not provided in excess.

In some embodiments, the molar ratio of the source of iodine to the reactive agent in the oral dosage form exceeds the corresponding molar ratio for the reaction(s) to form molecular iodine. In some embodiments, the molar ratio of the source of iodine to the reactive agent in the oral dosage form exceeds the corresponding molar ratio for the reaction(s) to form molecular iodine by at least about 25%, preferably by an amount in the range of about 25% to 1000%, more preferably by an amount in the range of about 50% to 500%, and most preferably by an amount in the range of about 50% to 300%.

In some embodiments, the molar ratio of the reactive agent to the source of iodine in the oral dosage form exceeds the corresponding molar ratio for the reaction(s) to form molecular iodine. In some embodiments, the molar ratio of the reactive agent to the source of iodine in the oral dosage form exceeds the corresponding molar ratio for the reaction(s) to form molecular iodine by at least about 25%, preferably by an amount in the range of about 25% to 1000%, more preferably by an amount in the range of about 50% to 500%, and most preferably by an amount in the range of about 50% to 300%.

In some preferred embodiments, the oral dosage form further comprises one or more catalysts for the reaction(s) to form molecular iodine. By further increasing the rate of reaction, the source of iodine and reactive agent have even less time to react with other materials in the stomach, such as food that has been ingested. This results in less variability for the amount of molecular iodine that is produced. It also potentially reduces the rate of side effects and drug interactions, particularly for a component that is not provided in excess.

Examples of catalysts will depend on the type of reaction. For example, for a reaction between an iodate salt and an iodide salt, exemplary catalysts include carboxylates, phosphates, and peroxidases. Examples of suitable carboxylates include acetates, lactates, citrates, glycolates, succinic, malates, adipates and amino acids. Examples of suitable phosphates include soluble salts of phosphoric acid including sodium or potassium phosphate. Examples of suitable peroxidases include horseradish peroxidase, soybean peroxidase, lactoperoxidase, myerloperoxidase, NADH peroxidase, NDAPH peroxidase, fatty-acid peroxidase, cytochromes-c peroxidase, catalase, glutathione peroxidase, L-ascorbate peroxidase, phospholipid-hydroperoxide manganese peroxidase, lignin peroxidase, peroxiredoxin, versatile peroxidase, glutathione amide-dependent peroxidase, bromide peroxidase, dye decolorizing peroxidase, prostamide/prostaglandin F2α synthase, catalase-peroxidase, and hydroperoxy fatty acid reductase. Other examples of catalysts will be apparent to those skilled in the art.

Typical amounts of the catalyst in the instant oral dosage form range from about 1% to about 50% by weight, from about 5% to about 40% by weight, or from about 10% to about 30% by weight. In embodiments, the dosage form comprises about 1-5%, 1-10%, 1-20%, 1-30%, 1-40%, 5-10%, 5-20%, 5-30%, 5-40%, 5-50%, 10-20%, 10-30%, 10-40%, 10-50%, 20-30%, 20-40%, 20-50%, 30-40%, 30-50%, or about 40-50% of the catalyst.

In some preferred embodiments, the oral dosage form further comprises one or more scavenger that reacts with an excess component (i.e., the source of iodine or the reactive agent that was provided in excess relative to the molar ratio for the reaction to form molecular iodine). Scavengers for use in the instant oral dosage form are typically thiols. Examples of suitable scavengers for the instant oral dosage form include proteins comprising a sulfhydryl group, cysteine, glutathione, ergothioneine, and mixtures thereof. By reacting with the excess component, a scavenger limits potential side effects or drug interactions that could otherwise occur due to the reactions of the excess component with body tissues or other substances in the digestive tract.

In some preferred embodiments, the scavenger is selected from the group consisting of cysteine and glutathione. One particular advantage of cysteine and glutathione is that these thiols react slowly with iodate as demonstrated by Hird et al. (F J R Hird and J R Yates, “The Oxidation of Protein Thiol Groups by Iodate, Bromate and Persulphate” Biochemistry Journal (1961) 80:612-616) Cysteine, glutathione, or a combination thereof can therefore be used in an oral dosage form with a source of iodate such that the slow rate of reaction between iodate and cysteine and/or glutathione at low pH allows the reaction between the iodate and the source of iodine or the reactive agent to consume a larger fraction of the iodate than would be the case if the reaction between iodate and cysteine and/or glutathione were faster.

In some non-limiting embodiments, the scavenger is a sugar polymer such as starch, beta-glucans (e.g., cellulose), hemicellulose (e.g., hexosan and pentosane), lignin, xanthan, fructans (e.g., inulin), polyuronide (e.g., pectin, alginic acids, sodium alginate, potassium alginate, ammonium alginate, calcium alginate, propylene glycol alginate, agar, and carrageen), raffinose, xylose, polydextrose, and lactulose. These would be targeted for release in the small intestine by enterically coating them as to not allow for their competition for reactants in the stomach. Some preferred forms are cellulose and lignin, which, due to their indigestible nature, react with iodine compounds and remove them from the digestive system.

As will be evident to those skilled in the art, the amount of the scavenger in the instant oral dosage form will vary based on the amount of the material to be scavenged and other factors. Typical, but not limiting, amounts of the scavenger in the oral dosage form range from about 0.1% to 20% by weight, from about 0.25% to 10% by weight, or from about 0.5% to 5% by weight. In other embodiments, the oral dosage form comprises about 0.1-0.25%, 0.1-0.5%, 0.1-5%, 0.1-10%, 0.25-0.5%, 0.25-5%, 0.25-20%, 0.5-10%, 0.5-20%, 5-10%, 5-20%, or about 10-20% of the scavenger by weight.

In some embodiments, the scavenger is a delayed release scavenger such as an enterically coated scavenger. In some embodiments, for example, a scavenger is an enterically coated xanthan salt, which binds to excess iodide in the small intestine, thus rendering the excess iodide not bioavailable. Beneficially, such delayed-release scavengers, limit absorption of the unreacted excess component from the intestines. This is particularly important for iodide, which is pumped from the intestines into the blood stream via sodium iodide symporter (NIS) pumps. In some embodiments, enteric coatings comprise a polymethacrylate, for example, methacrylic acid copolymer dispersion, methacrylic acid copolymer type A, or combinations thereof. In some embodiments, enteric coatings comprise a cellulose-based polymer, for example cellulose acetate phthalate (CAP), cellulose acetate trimellitate (CAT), cellulose acetate succinate (CAS), hydroxypropyl methylcellulose phthalate (HPMCP), hydroxypropyl methylcellulose acetate succinate (HPMCAS), or combinations thereof. In some embodiments, enteric coatings comprise a polyvinyl derivative, for example polyvinyl acetate phthalate (PVAP). In some embodiments the enteric coatings comprise copolymers, for example half esters of copolymerisate of styrene and maleic acid, copolymerisate of vinyl ether and maleic acid, copolymerisate of vinyl acetate and crotonic acid, or combinations thereof.

In embodiments, the dosage form may comprise one or more pH adjustment agents. Examples of pH adjustment agents may be any pharmaceutically acceptable salt that can react with stomach acid to increase the pH to above 4.0. Examples include but are not limited to sodium, potassium, calcium and magnesium salts of carbonic acid and hydrogen carbonic acid. They also include calcium and magnesium hydroxide.

In an aqueous environment, molecular iodine reaches an equilibrium with other iodine species, such as triiodide. In particular, iodide reacts with molecular iodine to form triiodide. The oral dosage form may further include one or more hydrophobic components that can dissolve molecular iodine and remove it from the aqueous phase. This removes the molecular iodine from the equilibrium with triiodide and can reduce the amount of triiodide that remains in the aqueous phase. In some embodiments, addition of a lipid excipient to the composition assures that there is sufficient hydrophobic material collocated with the source of iodine and the reactive agent to dissolve the molecular iodine that is created. Thus, the proportion of molecular iodine that is created can be increased for a given amount of total iodine in the oral dosage form. Note that many lipid excipients are liquids at room temperature and so liquid dosage forms may be preferred in certain embodiments. In some preferred embodiments, the lipid excipient is saturated to limit the reaction with molecular iodine. Examples of lipid excipients include oils, fatty acids (e.g., C8 to C22 fatty acids, such as caprylic acid, capric acid, palmitic acid, stearic acid, oleic acid, linoleic acid, arachidic acid, and behenic acid), medium-chain triglycerides (e.g. caprylic triglyceride, capric triglyceride), and long-chain triglycerides (e.g., corn oil, olive oil, sesame seed oil, soybean oil, peanut oil). The use of some lipid excipients can direct a large percentage of molecular iodine to be absorbed from the gastro-intestinal (GI) tract into the circulatory or lymphatic systems via a particular path based on the nature of the lipid excipient in which the molecular iodine is dissolved. In some preferred embodiments, the use of lipid excipients that are absorbed from the GI tract after the stomach, such as in the small intestines. Examples of such lipid excipients include medium-chain triglycerides and long-chain triglycerides. This route of absorption of molecular iodine may help to maintain the stability of molecular iodine within the body by avoiding absorption into the bloodstream and/or processing by the liver. Some lipid excipients can aid in the absorption of molecular iodine into the lymphatic system, rather than into the blood stream, thus affecting the bioavailability of the molecular iodine.

The oral dosage form may be any form suitable for ingestion, e.g., in the form of a tablet, powder, powder for solution, liquid filled capsule, chewable tablet, granule, capsule syrup, powder for suspension, liquid solution, extended release tablet, lozenge, troche, and extended release capsule, or wafer. Without a loss of generality, the embodiments described herein are preferably in the form of a tablet. Other forms will be evident to those skilled in the art.

The oral dosage form may include one or more solid carriers/diluents that include, but are not limited to, a gum, a starch (e.g. corn starch, pregelatinized starch), a sugar (e.g., lactose, mannitol, sucrose, dextrose), a cellulosic material (e.g. microcrystalline cellulose), an acrylate (e.g. poly(methyl acrylate)), calcium carbonate, magnesium oxide, talc, or mixtures thereof.

The oral dosage form may also further comprise one or more binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hydroxypropyl methylcellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

The oral dosage form can be formed by any suitable method as known in the art such as, for example, by mixing, granulating, or tablet-forming processes, as are well understood in the art. The active ingredients are often mixed with excipients that are pharmaceutically acceptable and compatible with the active ingredients. For oral administration, the active ingredients as described herein are mixed with one or more additives customary for this purpose, such as vehicles, stabilizers, or inert diluents, and converted by customary methods into suitable forms for administration, such as tablets, coated tablets, hard or soft gelatin capsules, or other suitable solid dosage forms.

As used herein the term “excipient” refers to a pharmaceutically acceptable ingredient that is commonly used in the pharmaceutical technology for preparing granule and/or solid oral dosage formulations. Examples of categories of excipients include, but are not limited to, binders, disintegrants, lubricants, glidants, stabilizers, fillers and diluents. One of ordinary skill in the art may select one or more of the aforementioned excipients with respect to the particular desired properties of the granule and/or solid oral dosage form by routine experimentation and without any undue burden. The amount of each excipient used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2000).

A tablet can suitably be prepared by any method as known in the art. For instance, the solid oral dosage form ingredients that are targeted for the small intestine (e.g., cellulosics, unsaturated fatty acids) are mixed and granulated using dry mixing or by spray drying following mixture of the ingredients with an appropriate solvent. The resulting granules are then coated with an enteric coating. In a separate process, ingredients targeted for the stomach and used to create a sustained release formulation are mixed and granulated, e.g., using dry mixing or by spray drying following combination of the ingredients with a solvent. The resulting granules are then coated with an enteric coating, which can be a combination of one or more of the following: polyethylene, polyvinyl chloride, methyl acrylate-methacrylate copolymer, ethyl Cellulose, carnauba wax, stearyl alcohol, stearic acid, polyethylene glycol, polyethylene glycol monostearate, triglycerides, methyl cellulose, hydroxyethyl cellulose, hydroxypropyl methylcellulose, sodium carboxymethyl cellulose, carboxypolymethylene, sodium alginate, and galactomannans. In a separate process, the solid oral dosage form ingredients that are targeted for the stomach (e.g., iodide salt, iodate salt) are mixed and granulated using dry mixing or using a spray dry method following combination with a solvent. In some embodiments, the ingredients targeted for the stomach are dissolved in a granulation solvent to assist in their homogenous distribution throughout the solid dosage form. This enables the ratio between the iodide and iodate salts to be in a desired ratio for samples of 1 mg or more throughout the tablet. Two to three of these granulated mixtures are then blended together. The resulting mixture can be formed into tablets using a tablet press. Coating can be accomplished by pan spray, supercell, ACCELA-COTA (Thomas Engineering, Inc.; Hoffman Estates, Ill.), or any other method familiar to those skilled in the art of tablet making.

Film formers are selected such that they are soluble in the chosen solvent and meet the desired dissolution characteristics of the environment where the film is designed to dissolve. Examples of film formers that can be used for the enterically coated ingredients, include cellulose acetate phthalate (CAP), cellulose acetate trimellitate (CAT), hydroxypropyl methylcellulose phthalate (HPMCP), EUDRAGIT L, and EUDRAGIT S. Examples of film formers that dissolve in SGF (and in stomach acid) are hydroxypropyl methylcellulose (HPMC), methyl hydroxyethyl cellulose (MHEC), EUDRAGIT E, and EUDRAGIT RL and RS.

Examples of combinations that can be used to generate molecular iodine in SGF include (1) oxidants comprising iodine (iodine oxidizer) combined with reductants comprising iodine (iodine reducer), (2) sources of ferric iron (e.g., ferric salts) plus sources of iodide (e.g., iodide salts), (3) sources of iodide plus oxidants, and (4) sources of iodate plus reductants.

Examples of solid oral dosage forms are described in Table 1 in Example 1. This table lists only select ingredients, and does not include excipient ingredients such as binders, colorants, plasticizers, etc. The amounts of those inert ingredients will typically depend on specific manufacturing or marketing requirements that are well known to those skilled in the art.

### EXAMPLES

Turning now to consideration of the Examples, Example 1 provides ingredients for exemplary oral dosage forms.

Example 2 provides a study that demonstrated that hydrophilic iodide species are reduced while molecular iodine is increased by increasing the ratio of potassium iodate to potassium iodide above 0.20.

Example 3 provides a study that demonstrates the significant anti-proliferative and apoptotic effects of molecular iodine on two human breast cancer cell lines and one human fibrocystic breast cell line relative to human mammary epithelial cells from a healthy donor. The results indicate that molecular iodine targets breast cancer and fibrocystic breast cells selectively and exhibits anti-proliferative effects in these undesirable breast disease cells compared to human mammary epithelial cells.

The following examples are put forth to provide those of ordinary skill in the art with a complete disclosure and description of how the oral dosage form, its components, active ingredients, excipients, and the like, are prepared and evaluated, along with related methods, and are intended to be purely exemplary. Thus, the examples are in no way intended to limit the scope of what the inventors regard as their invention. There are numerous variations and combinations, e. g., component concentrations, desired solvents, solvent mixtures, antioxidants, and other mixture parameters and conditions that may be employed to optimize characteristics of the oral dosage form such as purity, yield, stability, and the like. Such are considered as well within the scope of the present disclosure.

### Example 1

### Oral Dosage Forms

Oral dosage forms as provided herein are prepared as described in examples F1-F6 below. Combinations of components from each of examples F1 to F6 represent exemplary embodiments of this disclosure. Such combinations are made by selecting an iodine source and reactive agent that react to produce molecular iodine at a total iodine content within a range as described herein. Additional embodiments further comprise one or more ingredients from the group consisting of a scavenger, a catalyst, a pH control agent, a pH buffer agent, an absorption matrix, and an enteric coating. These selected ingredients are then combined with one or more items from the group consisting of a film former, a solvent, a plasticizer, a binder, a lubricant, a colorant, an opaquant, and an extender to produce a solid dosage form in accordance with the teachings herein.

Note that the ingredient quantities listed in Table 1, combinations thereof, and other quantities described in this application refer to the quantities for each solid oral dosage form, regardless of whether a single solid oral dosage form is prepared or multiple solid oral dosage forms are prepared in a batch. If a batch of multiple solid oral dosage forms is prepared from a single mixture of ingredients, the quantity of each ingredient is scaled based on the quantity of solid oral dosage forms produced.

TABLE 1
Examples of ingredients for a solid oral dosage form

| | Example F1 | Example F2 | Example F3 | Example F4 | Example F5 | Example F6 |
|---|---|---|---|---|---|---|
| Iodine source | 8.0 mg | 50 mg | 13.08 mg | 12 mg | 19.62 mg | 120 mg |
| | potassium | potassium | potassium | potassium | potassium | potassium |
| | iodide | iodide | iodide | iodide | iodide | iodide |
| Reactive agent | 2.56 mg | 19.2 mg | 7.06 mg | 4.3 mg | 7.61 mg | 61.6 mg |
| | potassium | potassium | potassium | potassium | potassium | potassium |
| | iodate | iodate | iodate | iodate | iodate | iodate |
| Scavenger | | 50 mg | — | 10 mg | | 80 mg L- |
| | | cysteine | | glutathione- | | glutathione |
| Catalyst | — | — | 5 mg sodium | 10 mg | 10 mg | 80 mg |
| | | | bromide- | disodium | potassium | sodium |
| | | | | phosphate | lactate | phosphate |
| pH control | — | 6 mg | — | — | — | — |
| agent | | sodium | | | | |
| | | carbonate | | | | |
| pH buffer | — | — | — | 500 mg | 400 mg | 300 mg Mg |
| agent | | | | calcium | calcium | carbonate |
| | | | | carbonate | carbonate | |
| | | | | | 100 mg | |
| | | | | | magnesium | |
| | | | | | hydroxide | |
| Absorption | — | — | 100 mg alpha- | — | 100 mg | 50 mg |
| matrix | | | cyclodextrin | | hydroxypropyl | starch |
| | | | | | beta | |
| | | | | | cyclodextrin | |
| Enteric coating | — | — | 5 mg stearic | — | 7.5 mg stearic | — |
| | | | acid (5%) and | | acid (5%) and | |
| | | | 20 mg sodium | | 22.5 mg | |
| | | | carboxymethyl | | sodium | |
| | | | cellulose | | carboxymethyl | |
| | | | (20%) | | cellulose | |
| | | | | | (15%) | |
| Molar ratio of | 4.0 | 3.4 | 2.4 | 3.6 | 3.3 | 2.5 |
| iodide:iodate | | | | | | |
| Total iodine | 7.6 | 49.6 | 14.2 | 11.7 | 19.5 | 128.3 |
| (mg) | | | | | | |

### Example 2

### Study of Iodine Species

A study was performed to assess the effect of an oil-water mixture on relative proportions of species of iodine that were formed in a reaction of anhydrous potassium iodide and potassium iodate. A 100 mM solution of potassium iodide was produced and was mixed with successively diluted solutions of potassium iodate in the range of 100 mM to 1.56 mM. For each mixture, 1.00 mL of the potassium iodide solution was mixed with 0.80 mL of the potassium iodate solution to create mixtures with ratios of potassium iodate to potassium iodide in the range of 0.0125 to 0.80.

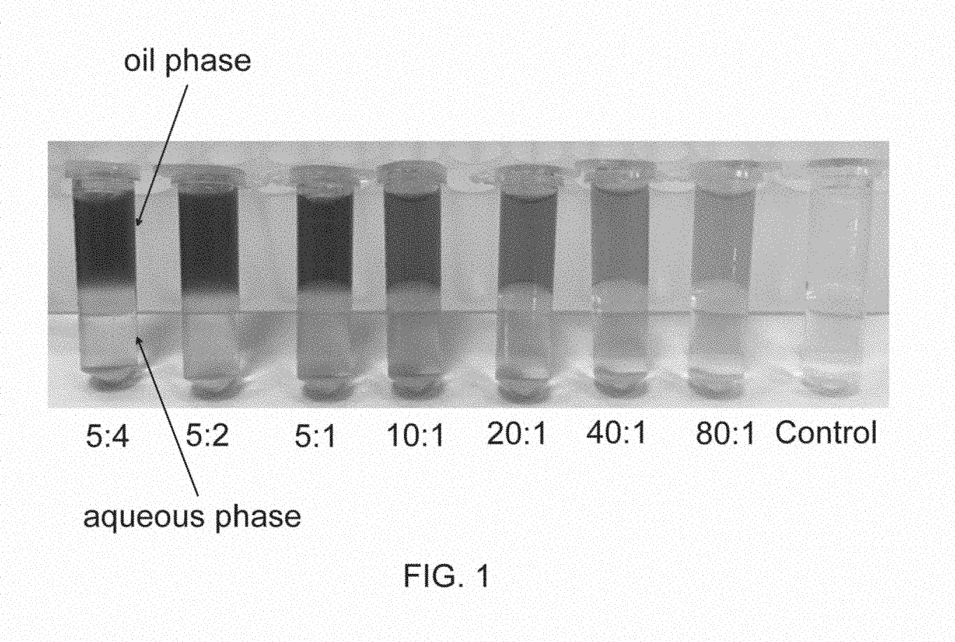

To each mixture was added 1.00 mL caprylic capric triglyceride, which is a medium-chain triglyceride, and 0.20 mL 1 M hydrochloric acid. The resulting water phase of this oil-water mixture is an acidic solution of 0.1 M HCl, which simulates the acidic pH of the stomach. Each of the resulting oil-water mixtures was mixed with a vortex mixer at 10000 rpm for 30 seconds and then allowed to settle. Images of the resulting solutions are shown in FIG. 1.

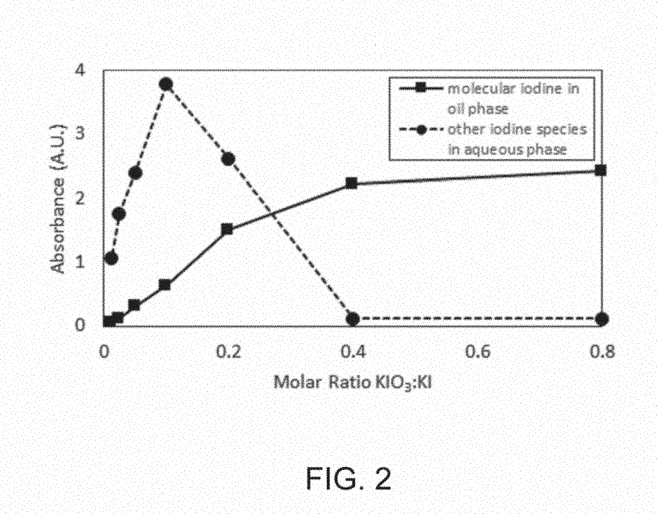

Since molecular iodine is hydrophobic and other prevalent iodine species are hydrophilic, the relative proportion of molecular iodine and other iodine species was able to be quantified by measuring the amount of iodine species in the oil and water phases of the resulting mixture. The optical absorbance of each sample was then measured. Absorbance was measured at a wavelength of 480 nm to evaluate the relative amount of molecular iodine in the oil phase. Absorbance was measured at a wavelength of 352 nm to evaluate the relative amount of iodide, polyiodide (e.g. triiodide), and other hydrophilic iodine species in the water phase. The resulting absorbance measurements are shown in FIG. 2. While the corresponding molar ratio of potassium iodate to potassium iodide for the reaction(s) to form molecular iodine is 0.20, the absorbance measurements clearly show that this ratio produces a large amount of residual hydrophilic iodine species, such as iodide and triiodide. To minimize the amount of iodide and triiodide, higher ratios of potassium iodate to potassium iodide are preferred. For example, the ratios of 0.40 and 0.80 show almost no residual hydrophilic iodine species in FIG. 2. Beneficially, these higher ratios also produce slightly more molecular iodine than the ratio of 0.20.

### Example 3

### Iodine Effects on Breast Cancer and Fibrocystic Breast Cell Lines

An in vitro study was performed to assess the effect of molecular iodine in breast cancer and fibrocystic breast cell lines relative to the effects in human mammary epithelial cells (HMEC)(“primary cells”). The study measured gene expression for key markers in each of the cell lines following in vitro addition to the cells of cell media mixtures that comprised iodine species.

Six cell media mixtures were formed for each cell line by mixing cell culture media (DMEM from Life Technologies, Thermo Fisher Scientific, Waltham, Mass., for the MCF7 and MDA-MB231 cell lines; MEBM from Life Technologies, Thermo Fisher Scientific, Waltham, Mass. for the MCF10A cell line; and MEGM from Lonza, Inc., Allendale, N.J., for the primary cells) with concentrated water-based solutions of iodine species at concentrations suitable to achieve the molecular iodine concentrations listed in Table 2. Concentrations of molecular iodine were measured with an iodine portable photometer (model H196718 Hanna Instruments, Woonsocket, R.I.).

TABLE 2
Cell media mixtures

| Cell | | Molecular iodine | |
|---|---|---|---|
| Media | Iodine species added to cell culture | concentration of | |
| Mixture | media to form cell media mixture | cell media mixture | |
| CM1 | none | 0 | micromolar |
| CM2 | 5% Lugol's solution (Ricca | 5 | millimolar |
| | Chemical, Arlington, TX) | | |
| CM3 | potassium iodide (Spectrum Chemicals | 1 | micromolar |
| | and Laboratory Products, New | | |
| | Brunswick, NJ) | | |
| CM4 | potassium iodate (Spectrum Chemicals | 5 | micromolar |
| | and Laboratory Products, New | | |
| | Brunswick, NJ) | | |
| CM5 | potassium iodide and potassium | 10 | micromolar |
| | iodate mixed at a molar ratio of 5:1 | | |
| CM6 | potassium iodide and potassium | 30 | micromolar |
| | iodate mixed at a molar ratio of 5:1 | | |
| CM7 | potassium iodide and potassium | 5 | micromolar |
| | iodate mixed at a molar ratio of 5:1 | | |

Samples of each of the six cell media mixtures CM1 to CM6 were combined individually with each of four cell lines: MCF7, MDA-MB231, MCF10A, and primary cells. These cell lines are representative of two common breast cancer sub-types and of fibrocystic breast tissue. The two breast cancer cell lines evaluated in the study were MCF7 cells (ATCC HTB-22, American Type Culture Collection, Manassas, Va.) from a luminal A breast cancer subtype and MDA-MB231 cells (ATCC HTB-26, American Type Culture Collection, Manassas, Va.) from a triple-negative breast cancer subtype. The fibrocystic breast tissue cell line evaluated in the study was MCF10A (ATCC CRL-10317, American Type Culture Collection, Manassas, Va.), an immortalized human mammary epithelial cell line derived from fibrocystic breast tissues of a 36-year old female Caucasian. Primary cells (human mammary epithelial cells CC-2551 from Lonza) were isolated from adult female breast tissue and evaluated as an internal control.

As mentioned in the previous paragraph, each of the four cell lines described in the previous paragraph was added to a sample of each of the six cell media mixtures CM1 to CM6. As an untreated control, one sample for each cell line was mixed with media only. Real-time reverse transcription polymerase chain reaction (qRT-PCR) was used to measure several key mRNA markers for apoptosis and cell growth in breast tissue for each combination of cell line and cell media mixture. The measured markers for cell growth were Cyclin D1, Cyclin E1, Cyclin B1, CDK2, and CDKN1B. The measured markers for apoptosis were BCL2 and Caspase 3. Cyclin D1 is required for progression through the G1 phase of the cell cycle. Cyclin E1 is required for cell cycle G1/S transition. Cyclin B1 is involved in the early events of mitosis, such as chromosome condensation, nuclear envelop breakdown, and spindle pole assembly. CDK2 is required for the transition from G1 to S phase and binding with Cyclin A is required to progress through the S phase. CDKN1B binds to and prevents the activation of cyclin E-CDK2 or Cyclin D-CDK4 complexes, and thus controls the cell cycle progression at G1. BCL2 plays an important role in promoting cellular survival and inhibiting the actions of pro-apoptotic proteins. Caspase 3 plays a central role in the execution phase of cell apoptosis. The resulting data for experiments from adding each cell line to cell media mixture CM6 are presented in Tables 3 and 4. Table 3 presents the data normalized to the corresponding data when the cells are added to CM1, which does not contain any iodine species. Table 4 presents the data normalized to the corresponding data for the primary cells when added to CM6.

TABLE 3
Results of qRT-PCR gene expression measurements for cells
added to cell media mixture CM6 normalized to expression
measurements for cells added to cell media mixture CM1.

| | MCF10A | MCF7 | MDA-MB231 |
|---|---|---|---|
| | cell line | cell line | cell line |
| Markers associated | | | |
| with cell cycle | | | |
| regulation or | | | |
| proliferation | | | |
| Cyclin D1 | 4 | 3.8 | 6.5 |
| Cyclin E1 | >10 | 8.5 | 6.2 |
| Cyclin B1 | 0.88 | 0.85 | 0.6 |
| CDK2 | 1.22 | 0.9 | 4.1 |
| CDKN1B | not | 3.23 | 3.5 |
| | measured | | |
| Markers associated | | | |
| with apoptosis | | | |
| BCL2 | >10 | 9.45 | 4.5 |
| Caspase 3 | 3.9 | 7.1 | 2.0 |

TABLE 4
Results of qRT-PCR gene expression measurements for cells added
to cell media mixture CM6 normalized to corresponding measurements
for the control cell line added to cell media mixture CM6.

| | ratio of | ratio of | ratio of |
|---|---|---|---|
| | expression for | expression for | expression for |
| | MCF10A cell | MCF7 cell | MDA-MB231 |
| | line to | line to | cell line to |
| | expression | expression | expression |
| | for the control | for the control | for the control |
| | cell line | cell line | cell line |
| Markers associated | | | |
| with cell cycle | | | |
| regulation or | | | |
| proliferation | | | |
| Cyclin D1 | 7.2 | 6.8 | 11.6 |
| Cyclin E1 | >5.3 | 4.5 | 3.3 |
| Cyclin B1 | 0.36 | 0.35 | 0.25 |
| CDK2 | 0.77 | 0.57 | 2.6 |
| CDKN1B | not measured | 2.9 | 3.2 |
| Markers associated | | | |
| with apoptosis | | | |
| BCL2 | >1.6 | 1.5 | 0.7 |
| Caspase 3 | 6.3 | 11.5 | 3.2 |

Gene expression analysis using qRT-PCR further confirmed that key cell cycle genes responsible for controlling G1-S phase transition were significantly up-regulated upon molecular iodine treatment. Data from these studies indicated that molecular iodine has potent inhibitory effects on cell growth in both breast cancer and FBC. The data also showed a dramatic increase in cell death in breast cancer cell lines used in the study and in cells derived from fibrocystic breast tissue, particularly relative to the cell death observed for human mammary epithelial cells. In the breast cancer and fibrocystic cell lines, gene expression analysis using quantitative RT-PCR demonstrated that cell cycle genes controlling G1-S phase transition were largely up-regulated. No significant changes were seen in Cyclin B expression levels which suggests that cells were arrested before entry into cell division. Expression of nuclear hormone receptors PPAR-α and PPAR-γ was up-regulated. BCL-2, an inhibitor of cell death, was increased, while expression of caspase-3 was decreased. This indicates that molecular iodine can induce cell death through activation of caspase-independent apoptosis.

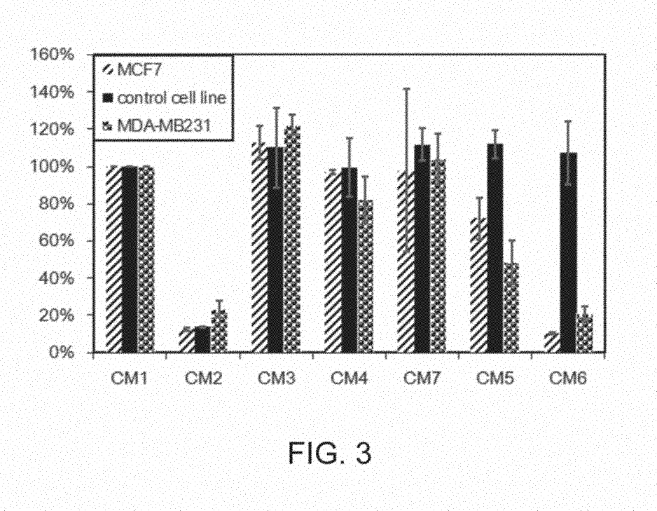

Cell viability for each of the four cell lines referenced supra was measured by a cell proliferation assay (CellTiter96AQᵤₑₒᵤₛ One Solution Cell Proliferation Assay, Promega, Madison, Wis.) 24 hours after adding cells to each of the seven cell media mixtures CM1 to CM7. The resulting data are presented in FIG. 3 normalized to the cell viability 24 hours after adding cells from the same cell line to cell media alone (i.e., CM1). These data demonstrate the effect of molecular iodine on cell proliferation for two breast cancer derived cell lines (i.e., MCF7 and MDA-MB231), a fibrocystic breast cell line (i.e., MCF10A), and human mammary epithelial cells (i.e., primary cells). Breast cancer cell lines are more susceptible to molecular iodine treatment and induce rapid cell death compared to primary cells. For low concentrations of molecular iodine (e.g. CM7, cell viability is roughly the same as for the control cell media (i.e., CM1).

In treatment or prophylaxis of breast cancer or fibrocystic breast condition, it is beneficial and strongly preferable to have a drug that is highly selective for apoptosis and cell proliferation between the cancerous or fibrocystic cells and the healthy human mammary epithelial cells. This allows the population of undesirable cells to be selectively reduced while having little, no, or less impact human mammary epithelial cells. The combination of our qRT-PCR results and our cell viability measurements indicates that molecular iodine formed by the combination of iodide and iodate has such selectivity. This selectivity is not present for other iodine species, such as Lugol's solution (CM2), potassium iodide (CM3), and potassium iodate (CM4).

Results from qRT-PCR demonstrated that molecular iodine treatment significantly increased the mRNA levels of key regulators involved in cell cycle progression while decreasing the level of Cyclin B1. These results indicate potent inhibitory effects on cell cycle progression in breast cancer cells and fibrocystic cells in comparison to human mammary epithelial cells. Thus, molecular iodine triggers rapid cell cycle arrest, differentiation, and apoptotic induction in breast cancer and fibrocystic cells but the impact on human mammary epithelial cells is significantly less than that for the breast cancer and fibrocystic cells.

Cell viability measurements demonstrated that cell media mixtures of iodide and iodate at concentrations of at least 10 micromolar of molecular iodine (e.g. CM5 and CM6) have significant selectivity for apoptosis in cancer cell lines in comparison with apoptosis in human mammary epithelial cells. This selectivity is useful for the treatment of breast cancer cells while maintaining viability of non-cancerous breast cells.

The concentrations of molecular iodine demonstrated to induce apoptosis and suppress proliferation are similar for breast cancer cells and fibrocystic cells, which indicates that comparable levels of molecular iodine would be useful for treatment or prophylaxis of both conditions. Clinical trials have demonstrated that levels in the range of about 3 to about 12 mg of molecular iodine per day are effective for the treatment of fibrocystic breast condition (see, for example, J H Kessler, “The Effect of Supraphysiologic Levels of Iodine on Patients with Cyclic Mastalgia” The Breast Journal, 2004; 10(4) 328-336 and W R Ghent, B A Eskin, D A Low, L P Hill, “Iodine Replacement in Fibrocystic Disease of the Breast” CJS October 1993; 36(5) 453-459). The similarity in the response for breast cancer and fibrocystic cells and the selectivity data presented herein indicates that similar dosage levels would be appropriate for treatment or prophylaxis of breast cancer. For treatment of breast cancer, higher dosage levels would be warranted due to the need for a more aggressive approach in such cases. For these reasons, daily dosage of 3 to 100 mg per day, or preferably 6 to 50 mg per day, or preferably 6 to 30 mg per day could be administered in the oral dosage form described herein for prophylaxis or treatment of breast cancer.

The data presented in this Example 3 demonstrate the significant anti-proliferative and apoptotic effects of molecular iodine on two breast cancer cell lines and one fibrocystic breast cell line relative to human mammary epithelial cells from a healthy donor. The results indicate that molecular iodine targets breast cancer and fibrocystic breast cells selectively and exhibits anti-proliferative effects in these undesirable breast cells compared to human mammary epithelial cells.

### Embodiments

1. An oral dosage form comprising

a source of iodine, and

a reactive agent, wherein
(i) the total iodine in the oral dosage form is about 3 to 60 milligrams,
(ii) the source of iodine reacts with the reactive agent in simulated gastric fluid to form molecular iodine, and
(iii) the amount of at least one of the source of iodine or the reactive agent is present in excess of the stoichiometric amount required for the reaction to form molecular iodine.
2. The oral dosage form of embodiment 1, further comprising a source of carboxylate or phosphate.
3. The oral dosage form of the combined or separate embodiments 1-2, further comprising a catalyst that increases the rate of the reaction between the source of iodine and the reactive agent in (ii).
4. The oral dosage form of the combined or separate embodiments 1-3, further comprising a source of carboxylate.
5. The oral dosage form of the combined or separate embodiments 1-4, further comprising a source of phosphate.
6. The oral dosage form of the combined or separate embodiments 1-5, further comprising a scavenger that reacts with at least a portion of the excess in (iii).
7. The oral dosage form of the combined or separate embodiments 1-6, wherein the scavenger is a protein comprising a sulfhydryl group.
8. The oral dosage form of the combined or separate embodiments 1-7, wherein the scavenger comprises either cysteine or glutathione.
9. The oral dosage form of the combined or separate embodiments 1-8, wherein the rate of reaction between the source of iodine and the reactive agent is faster than the rate of reaction between the excess amount of the source of iodine or the reactive agent and the scavenger.
10. The oral dosage form of the combined or separate embodiments 1-9, wherein the scavenger has a pH-dependent rate of reaction wherein less than 30% of the reaction is completed after a two-hour acid stage as defined in U.S. Pharmacopeia <711> Method A for delayed-release dosage forms and a rate of reaction increases when subsequently placed in the buffer stage of that method.
11. The oral dosage form of the combined or separate embodiments 1-10, wherein the scavenger has a pH-dependent rate of reaction wherein less than 10% of the reaction is completed after a two-hour acid stage as defined in U.S. Pharmacopeia <711> Method A for delayed-release dosage forms and a rate of reaction increases when subsequently placed in the buffer stage of that method.
12. The oral dosage form of the combined or separate embodiments 1-11, wherein the scavenger has a pH-dependent rate of reaction wherein less than 5% of the reaction is completed after a two-hour acid stage as defined in U.S. Pharmacopeia <711> Method A for delayed-release dosage forms and a rate of reaction increases when subsequently placed in the buffer stage of that method.
13. The oral dosage form of the combined or separate embodiments 1-12, wherein the dosage form is an extended-release dosage form.
14. The oral dosage form of the combined or separate embodiments 1-13, wherein the dosage form releases no more than 5% of at least one of the source of iodine or the reactive agent in the first 20 minutes when tested according to U.S. Pharmacopeia <711>.
15. The oral dosage form of the combined or separate embodiments 1-14, wherein the dosage form releases no more than 25% of at least one of the source of iodine or the reactive agent in the first 20 minutes when tested according to U.S. Pharmacopeia <711>.
16. The oral dosage form of the combined or separate embodiments 1-15, wherein the dosage form is a delayed-release dosage form.
17. The oral dosage form of the combined or separate embodiments 1-16, wherein more than 10% of at least one of the source of iodine or the reactive agent remains undissolved after 2 hours in a test according to Method A for delayed-release dosage forms in U.S. Pharmacopeia <711>.
18. The oral dosage form the combined or separate embodiments 1-17, wherein more than 10% of a scavenger remains undissolved after 2 hours in a test according to Method A for delayed-release dosage forms in U.S. Pharmacopeia <711>.
19. The oral dosage form of the combined or separate embodiments 1-18, wherein more than 10% of a catalyst remains undissolved after 2 hours in a test according to Method A for delayed-release dosage forms in U.S. Pharmacopeia <711>.
20. The oral dosage form of the combined or separate embodiments 1-19, wherein the reaction between the source of iodine and the reactive agent in (ii) is an oxidation-reduction reaction.
21. The oral dosage form of the combined or separate embodiments 1-1-20, wherein the ratio of the source of iodine to the reactive agent exceeds, by at least 25%, the corresponding molar ratio for the reaction(s) to form molecular iodine.
22. The oral dosage form of the combined or separate embodiments 1-21, wherein the ratio of the source of iodine to the reactive agent exceeds, by 50% to 500%, the corresponding molar ratio for the reaction(s) to form molecular iodine.
23. The oral dosage form of the combined or separate embodiments 1-22, wherein the source of iodine and the reactive agent are mixed in a homogeneous distribution within the oral dosage form.
24. The oral dosage form of the combined or separate embodiments 1-23, wherein the source of iodine comprises iodide, and the reactive agent comprises iodate.
25. The oral dosage form of embodiment 24, wherein the molar ratio between the iodide and the iodate in the dosage form is in a range of 6.5:1 to 100:1.
26. The oral dosage form of embodiment 24, wherein the molar ratio between the iodide and the iodate in the dosage form is in a range of 10:1 to 50:1.
27. The oral dosage form of embodiment 24, wherein the molar ratio between the iodide and the iodate in the dosage form is in a range of 1:100 to 4:1.
28. The oral dosage form of embodiment 24, wherein the molar ratio between the iodide and the iodate in the dosage form is in a range of 1:50 to 3:1.
29. The oral dosage form of embodiment 24, wherein the molar ratio between the iodate and the iodide in the dosage form is in a range of 1:4 to 5:1.
30. The oral dosage form of embodiment 24, wherein the molar ratio between the iodate and the iodide in the dosage form is in a range of 3:10 to 5:1.
31. The oral dosage form of embodiment 24, wherein the molar ratio between the iodate and the iodide in the dosage form is in a range of 1:2 to 5:1.
32. The oral dosage form of embodiment 24, wherein the molar ratio between the iodate and the iodide in the dosage form is in a range of 1:4 to 1:1.
33. The oral dosage form of the combined or separate embodiments 1-23, wherein the source of iodine comprises iodide, and the reactive agent is selected from the group consisting of an iodate salt, hydrogen peroxide, a source of iodate, and a source of hydrogen peroxide.
34. The oral dosage form of embodiment 33, wherein the molar ratio between the iodide and the reactive agent in the dosage form is in a range of 6.5:1 to 100:1.
35. The oral dosage form of embodiment 33, wherein the molar ratio between the iodide and the reactive agent in the dosage form is in a range of 10:1 to 50:1.
36. The oral dosage form of embodiment 33, wherein the molar ratio between the iodide and the reactive agent in the dosage form is in a range of 1:100 to 4:1.
37. The oral dosage form of embodiment 33, wherein the molar ratio between the iodide and the reactive agent in the dosage form is in a range of 1:50 to 3:1.
38. The oral dosage form of embodiment 33, wherein the molar ratio between the reactive agent and the iodide in the dosage form is in a range of 1:4 to 5:1.
39. The oral dosage form of embodiment 33, wherein the molar ratio between the reactive agent and the iodide in the dosage form is in a range of 3:10 to 5:1.
40. The oral dosage form of embodiment 33, wherein the molar ratio between the reactive agent and the iodide in the dosage form is in a range of 1:2 to 5:1.
41. The oral dosage form of embodiment 33, wherein the molar ratio between the reactive agent and the iodide in the dosage form is in a range of 1:4 to 1:1.
42. The oral dosage form of the combined or separate embodiments 1-23, wherein the source of iodine comprises iodide and the reactive agent is a ferric salt.
43. The oral dosage form of the combined or separate embodiments 1-42, further comprising a pH control agent such that the effective pH of the oral dosage form is between 7.0 and 12.0.
44. The oral dosage form of the combined or separate embodiments 1-43, further comprising a pH buffer agent wherein the pH buffer agent adjusts the pH of 0.5 liter of simulated gastric fluid to a pH of 4.0-8.0.
45. The oral dosage form of the combined or separate embodiments 1-44, further comprising 300 mg to 1000 mg of a pH buffer agent.
46. The oral dosage form of the combined or separate embodiments 1-45, further comprising an absorption matrix that stabilizes molecular iodine in solution.
47. The oral dosage form of the combined or separate embodiments 1-46, wherein the total iodine in the oral dosage form is 6 to 50 milligrams.
48. The oral dosage form of the combined or separate embodiments 1-47, further comprising a lipid excipient.
49. The oral dosage form of the combined or separate embodiments 1-48, wherein the lipid excipient comprises a medium chain triglyceride or a long chain triglyceride
50. A method of treatment or prophylaxis of a condition or disease responsive to treatment or prophylaxis with iodine, comprising the steps of administering an oral dosage form as described in the combined or separate embodiments 1-49 at least once daily to a human patient for a period of at least 30 days.
51. The method of embodiment 50, wherein the condition or disease is breast cancer.
52. The method of the combined or separate embodiments 50-51, wherein the condition or disease is fibrocystic breast condition.
53. A method for fabricating an oral dosage form comprising the steps of
   (i) providing a source of iodine,
   (ii) providing a reactive agent, and
   (iii) combining the source of iodine and the reactive agent to form an oral dosage form with an effective pH above 7,
      wherein either the source of iodine or the reactive agent is provided in excess of the corresponding molar ratio for the reaction(s) to form molecular iodine.

1. An oral dosage form comprising
a source of iodine, and
a reactive agent, wherein
(i) the total iodine in the oral dosage form is 3 to 60 milligrams,
(ii) the source of iodine reacts with the reactive agent in simulated gastric fluid to form molecular iodine, and
(iii) the amount of at least one of the source of iodine or the reactive agent is present in excess of the stoichiometric amount required for the reaction to form molecular iodine.

2. The oral dosage form of claim 1, further comprising a source of carboxylate or phosphate.

3. The oral dosage form of claim 1, further comprising a catalyst that increases the rate of the reaction between the source of iodine and the reactive agent in (ii).

4. The oral dosage form of claim 1, further comprising a scavenger that reacts with at least a portion of the excess in (iii).

5. The oral dosage form of claim 4, wherein the scavenger is a protein comprising a sulfhydryl group.

6. The oral dosage form of claim 5, wherein the scavenger comprises either cysteine or glutathione.

7. The oral dosage form of claim 4, wherein the rate of reaction between the source of iodine and the reactive agent is faster than the rate of reaction between the excess amount of the source of iodine or the reactive agent and the scavenger.

8. The oral dosage form of claim 4, wherein the scavenger has a pH-dependent rate of reaction wherein less than 30% of the reaction is completed after a two-hour acid stage as defined in U.S. Pharmacopeia <711> Method A for delayed-release dosage forms and a rate of reaction increases when subsequently placed in the buffer stage of that method.

9. The oral dosage form of claim 1, wherein the dosage form is an extended-release dosage form.

10. The oral dosage form of claim 1, wherein the dosage form is a delayed-release dosage form.

11. The oral dosage form of claim 10, wherein more than 10% of at least one of the source of iodine or the reactive agent remains undissolved after 2 hours in a test according to Method A for delayed-release dosage forms in U.S. Pharmacopeia <711>.

12. The oral dosage form of claim 10, wherein more than 10% of a scavenger remains undissolved after 2 hours in a test according to Method A for delayed-release dosage forms in U.S. Pharmacopeia <711>.

13. The oral dosage form of claim 10, wherein more than 10% of a catalyst remains undissolved after 2 hours in a test according to Method A for delayed-release dosage forms in U.S. Pharmacopeia <711>.

14. The oral dosage form of claim 1, wherein the reaction between the source of iodine and the reactive agent in (ii) is an oxidation-reduction reaction.

15. The oral dosage form of claim 1, wherein a ratio of the source of iodine to the reactive agent exceeds, by at least 25%, a corresponding molar ratio for the reaction(s) to form molecular iodine.

16. The oral dosage form of claim 1, wherein
the source of iodine comprises iodide, and
the reactive agent comprises iodate.

17. The oral dosage form of claim 16, wherein a molar ratio between the iodide and the iodate in the dosage form is in a range of 6.5:1 to 100:1.

18. The oral dosage form of claim 16, wherein a molar ratio between the iodide and the iodate in the dosage form is in a range of 1:100 to 4:1.

19. The oral dosage form of claim 16, wherein a molar ratio between the iodide and the iodate in the dosage form is in a range of 1:50 to 3:1.

20. The oral dosage form of claim 1, wherein
the source of iodine comprises iodide, and
the reactive agent is selected from the group consisting of an iodate salt, hydrogen peroxide, a source of iodate, and a source of hydrogen peroxide.

21. The oral dosage form of claim 20, wherein a molar ratio between the iodide and the reactive agent in the dosage form is in a range of 6.5:1 to 100:1.

22. The oral dosage form of claim 20, wherein a molar ratio between the iodide and the reactive agent in the dosage form is in a range of 1:100 to 4:1.

23. The oral dosage form of claim 1, wherein the source of iodine comprises iodide and the reactive agent is a ferric salt.

24. The oral dosage form of claim 1, further comprising a pH control agent such that the effective pH of the oral dosage form is between 7.0 and 12.0.

25. The oral dosage form of claim 1, wherein the total iodine in the oral dosage form is 6 to 50 milligrams.

26. The oral dosage form of claim 1, further comprising a lipid excipient.

27. The oral dosage form of claim 26, wherein the lipid excipient comprises a medium chain triglyceride or a long chain triglyceride

28. A method of treatment or prophylaxis of a condition or disease responsive to treatment or prophylaxis with iodine, comprising the steps of
administering an oral dosage form as described in claim 1 at least once daily to a human patient for a period of at least 30 days.

29. The method of claim 28, wherein the condition or disease is breast cancer.

30. The method of claim 28, wherein the condition or disease is fibrocystic breast condition.

31. A method for fabricating an oral dosage form comprising the steps of
(i) providing a source of iodine,
(ii) providing a reactive agent, and
(iii) combining the source of iodine and the reactive agent to form an oral dosage form with an effective pH above 7,
wherein either the source of iodine or the reactive agent is provided in excess of the corresponding molar ratio for the reaction(s) to form molecular iodine.
